(19) United States
(12) Patent Application Publication
(10) Pub. No.: US 2012/0165617 A1
(43) Pub. Date: Jun. 28, 2012

(54) PATIENT ENABLED METHODS, APPARATUS, AND SYSTEMS FOR EARLY HEALTH AND PREVENTIVE CARE USING WEARABLE SENSORS

(75) Inventors: Guy Robert Vesto, Kildeer, IL (US); Rhett Alden, Seattle, WA (US)

(73) Assignee: General Electric Company, Schenectady, NY (US)

(21) Appl. No.: 12/979,603

(22) Filed: Dec. 28, 2010

(51) Int. Cl. A61B 5/00

(52) U.S. Cl. 600/300

(57) ABSTRACT

Certain examples provide systems, methods, and apparatus for patient-enabled early health and prevention. An example patient preventive health system includes a monitoring application interface to receive data from one or more sensors positioned with respect to a patient. The system includes a sensor data processor to process the received data from the one or more sensors to identify one or more readings based on the received data. The system includes an event analyzer to process the one or more readings to generate an event output. The system includes a patient notifier to notify the patient based on the event output. The system includes a biomarker transmitter to identify and transmit a biomarker to a clinical research cloud based on the one or more readings.

### FIELD

The present invention generally relates to a patient health prediction. In particular, the present invention relates to systems, methods, and apparatus for patient early health and preventive care using data from wearable sensors.

### BACKGROUND

Healthcare has become centered around electronic data and records management. Information systems in healthcare include, for example, healthcare information systems (HIS), radiology information systems (RIS), clinical information systems (CIS), and cardiovascular information systems (CVIS), and storage systems, such as picture archiving and communication systems (PACS), library information systems (LIS), and electronic medical records (EMR). Information stored may include patient medical histories, imaging data, test results, diagnosis information, management information, and/or scheduling information, for example. The content for a particular information system may be centrally stored or divided at a plurality of locations. Healthcare practitioners may desire to access patient information or other information at various points in a healthcare workflow. Availability of data also provides opportunities for healthcare analytics.

### BRIEF SUMMARY

Certain examples provide systems, methods, and apparatus for patient-enabled early health and prevention.

Certain examples provide a patient preventive health system including a monitoring application interface to receive data from one or more sensors positioned with respect to a patient. The system includes a sensor data processor to process the received data from one or more sensors to identify one or more readings based on the received data. The system includes an event analyzer to process one or more readings to generate an event output. The system includes a patient notifier to notify the patient based on the event output. The system includes a biomarker transmitter to identify and transmit a biomarker to a clinical research cloud based on the one or more readings.

Certain examples provide a tangible computer readable storage medium including executable program instructions which, when executed by a computer processor, cause the computer to implement patient the preventive health system. The system includes a sensor data processor to process the received data from one or more sensors to identify one or more readings based on the received data. The system includes an event analyzer to process one or more readings to generate an event output. The system includes a patient notifier to notify the patient based on the event output. The system includes a biomarker transmitter to identify and transmit a biomarker to a clinical research cloud based on the one or more readings.

Certain examples provide a computer-implemented method for providing patient-enabled early health and preventive care. The method includes suggesting, based on patient predisposal information related to at least one of a disease and a medical condition, a prevention plan for the patient. The method includes receiving, from one or more sensors positioned with respect to the patient, data related to patient health status. The method includes adjusting the prevention plan for the patient based on received data from the one or more sensors. The method includes applying one or more filters to the received data to identify one or more abnormal sensor readings. The method includes providing, based on review and consent by the patient, data regarding the one or more abnormal sensor readings to a clinical data aggregation and predictive analytics for further processing.

### BRIEF DESCRIPTION OF SEVERAL VIEWS OF THE DRAWINGS

FIG. 1 illustrates a flow diagram for an example method for patient-enabled early health and preventative care.

FIG. 2 illustrates an example patient-enabled early health and preventative care system.

FIG. 3 is a block diagram of an example processor system that may be used to implement systems, apparatus, and methods described herein.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF CERTAIN EXAMPLES

Although the following discloses example methods, systems, articles of manufacture, and apparatus including, among other components, software executed on hardware, it should be noted that such methods and apparatus are merely illustrative and should not be considered as limiting. For example, it is contemplated that any or all of these hardware and software components could be embodied exclusively in hardware, exclusively in software, exclusively in firmware, or in any combination of hardware, software, and/or firmware. Accordingly, while the following describes example methods, systems, articles of manufacture, and apparatus, the examples provided are not the only way to implement such methods, systems, articles of manufacture, and apparatus.

When any of the appended claims are read to cover a purely software and/or firmware implementation, in an embodiment, at least one of the elements is hereby expressly defined to include a tangible medium such as a memory, DVD, CD, Blu-ray, etc., storing the software and/or firmware.

Certain examples connect consumers (e.g., patients) to advancements in healthcare, such as in molecular medicine and clinical research relevant to their predisposed diseases (e.g., genetically, hereditarily, environmentally, etc., pre-disposed or inclined to suffer from). Furthermore, certain examples provide early warning systems, apparatus, and methods including guidance for a user to seek professional intervention. Certain examples provide an “early health” knowledge exchange clearinghouse for patients using smart phones and wearable sensors.

A worldwide explosion of mobile phones provides an untapped potential for wireless medicine. Additionally, an explosion of available data provides opportunities for “big data” analytics (e.g., Medical Quality Improvement Consortium (MQIC) analytics and/or other clinical decision support). Empowering the consumer and focusing on preventative care and early health can help reduce the overall cost of healthcare.

Furthermore, advancements in molecular medicine bring big data and information sharing challenges. Few have focused on how to distribute new discoveries and learning directly to consumers. Genetic testing has become affordable, but, as consumers are becoming more aware of diseases for which they are predisposed, they will also become more concerned about how to prevent and manage them. The amount of available information relevant to a patient's medical disposition and treatment is growing at a rate with which doctors cannot keep pace. In fact, more medical literature is published annually than a doctor can read in a lifetime. Certain examples identify and analyze these trends and enable knowledge sharing for early health and disease prevention.

A consumer (e.g., patient) who may have undergone genetic testing and/or have knowledge of family history may be aware of potential predisposed disease(s) that might develop later in his or her life, for example. The consumer, who may or may not have received professional counseling, may be concerned and want to take a more active role in preventing predisposed diseases from developing. However, staying educated and informed about the latest relevant advancements in medicine and preventive care is not easy or always possible. A large amount of information is available and is constantly evolving as new discoveries are shared. Advancements in mobile computing, cell phones, smart phones, etc., and wearable sensor technology can be used to enable consumers to monitor personal statistics, such as vital signs, heart functions, glucose, cancer biomarkers, etc. This monitoring helps enable the consumer to receive advanced warning if a disease is developing and/or to manage chronic disease, for example.

As diseases and preventive care methods are studied across patient populations, researchers and scientist can gain new insight. Unfortunately, these new discoveries and preventive care methods can take a long time to make their way into routine patient care (e.g., up to 17 years for evidence-based medicine). Smartphone sensors (such as skin patches for cardiac monitoring, biomarker skin tests, glucose testing, and/or other vital sign sensors, etc.) can serve as effective early warning systems. However, these types of mobile applications may be designed to look for specific conditions. Having access to a patient's complete medical history and genetic profile, in addition to the sensor data, can improve a physician's ability to identify a disease early and initiate an appropriate preventive measure(s). Certain examples help enable an ecosystem and clearinghouse to bring together sensor manufactures, clinical data aggregators and advanced analytics, clinical researchers, doctors, patients and health benefit plans, for example.

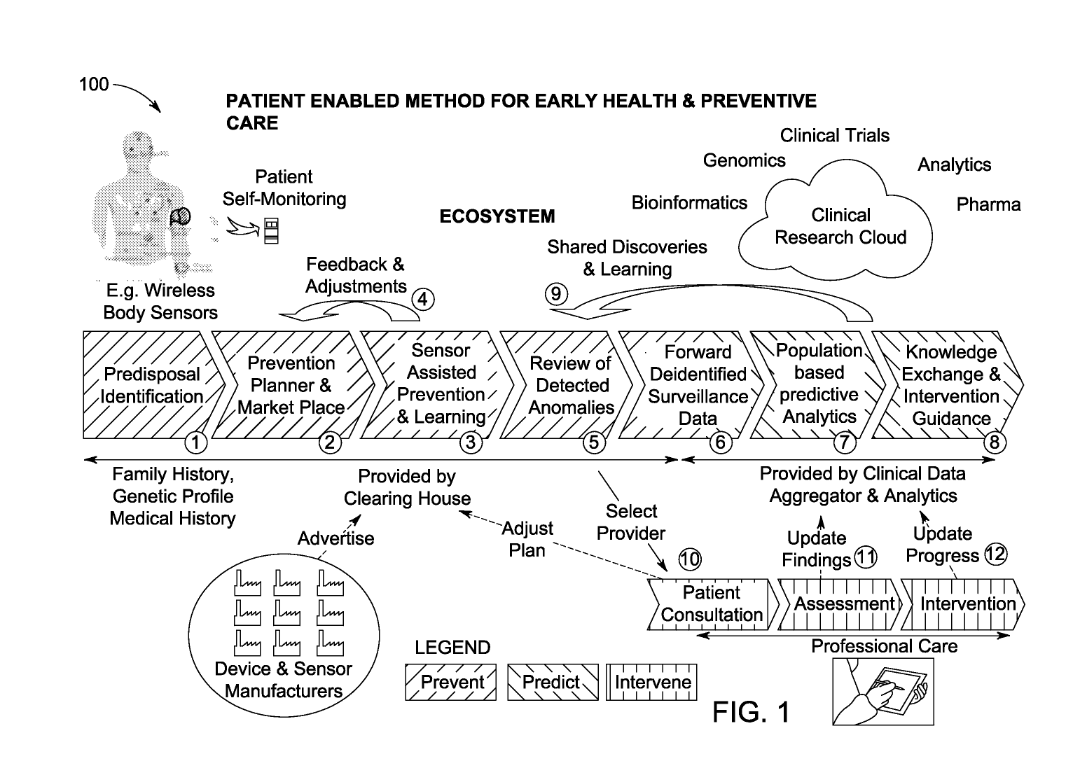

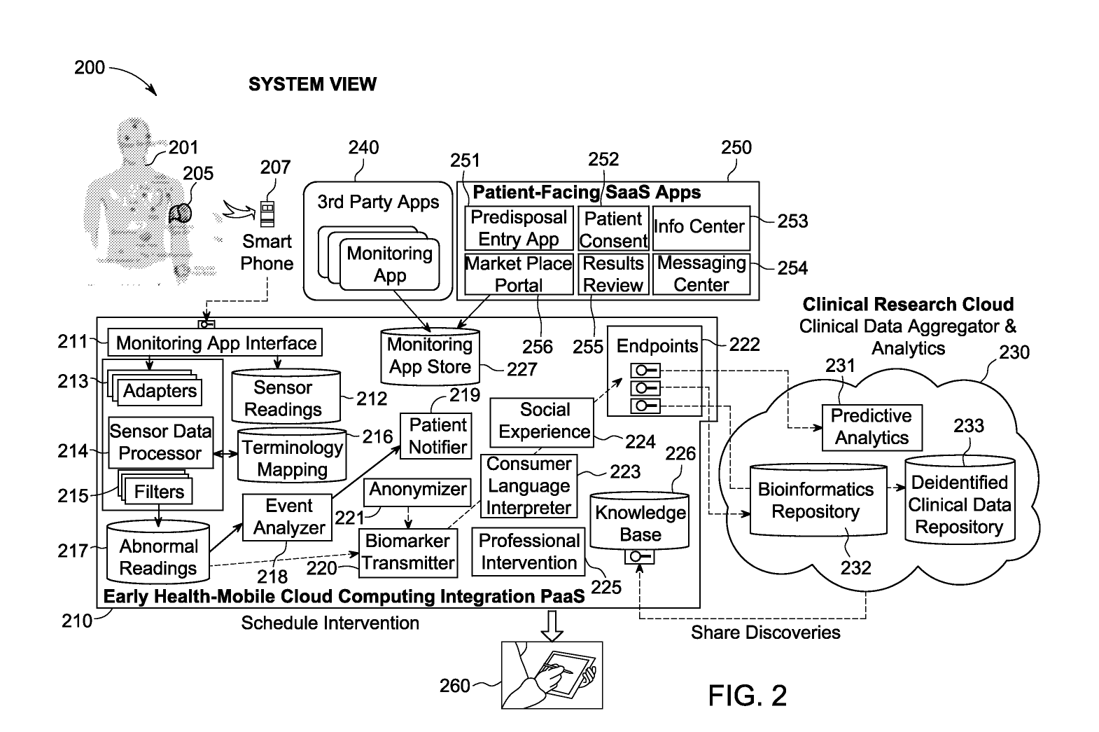

FIG. 1 represents a flow diagram representative of example machine readable instructions that can be executed to implement the example systems shown in FIG. 2 and/or portions of one or more of those and/or other systems. The example process(es) of FIG. 1 can be performed using a processor, a controller and/or any other suitable processing device. For example, the example process(es) of FIG. 1 can be implemented using coded instructions (e.g., computer readable instructions) stored on a tangible computer readable medium such as a flash memory, a read-only memory (ROM), and/or a random-access memory (RAM). As used herein, the term tangible computer readable medium is expressly defined to include any type of computer readable storage and to exclude propagating signals. Additionally or alternatively, the example process(es) of FIG. 1 can be implemented using coded instructions (e.g., computer readable instructions) stored on a non-transitory computer readable medium such as a flash memory, a read-only memory (ROM), a random-access memory (RAM), a cache, or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

Alternatively, some or all of the example process(es) of FIG. 1 can be implemented using any combination(s) of application specific integrated circuit(s) (ASIC(s)), programmable logic device(s) (PLD(s)), field programmable logic device(s) (FPLD(s)), discrete logic, hardware, firmware, etc. Also, some or all of the example process(es) of FIG. 1 can be implemented manually or as any combination(s) of any of the foregoing techniques, for example, any combination of firmware, software, discrete logic and/or hardware. Further, although the example process(es) of FIG. 1 are described with reference to the flow diagram of FIG. 1, other methods of implementing the process(es) of FIG. 1 can be employed. For example, the order of execution of the blocks can be changed, and/or some of the blocks described can be changed, eliminated, sub-divided, or combined. Additionally, any or all of the example process(es) of FIG. 1 can be performed sequentially and/or in parallel by, for example, separate processing threads, processors, devices, discrete logic, circuits, etc.

FIG. 1 illustrates a flow diagram for an example method 100 for patient-enabled early health and preventative care. At block 1, a consumer (e.g., a patient) enters or updates his or her disease pre-disposal information. This information can come from genetic testing and/or derived from family history and/or patient environmental information, for example. The information can be entered into a clinical information system workstation, electronic medical record (EMR), electronic health record (EHR), personal health record (PHR), etc., by the patient and/or by a healthcare practitioner, for example.

At block 2, based on the predisposal information, prevention plan(s), mobile sensor(s), and/or home health devices can be identified and suggest to the consumer. Sensor and device manufactures can advertise their products in a market place, for example, and the consumer can purchase a sensor or home health device and receive information regarding potential reimbursement from his or her health plan and/or employee benefits.

At block 3, in a self-monitoring phase, the consumer wears one or more sensors together with a smartphone and/or other type of home health monitoring device. The sensor(s) can be disposable, for example. One or more mobile and/or Web-based applications associated with these sensor(s) can be integrated with an information clearinghouse and transmit sensor readings to an associated system, for example.

At block 4, adjustments can be made to a prevention plan as the consumer learns more about his or her health status through the sensor-assisted self-monitoring.

At block 5, “intelligent” filters are able to separate normal sensor readings from abnormal ones. The consumer is notified as abnormal sensor readings are detected, for example.

At block 6, the consumer can then review abnormal readings and decide whether to authorize the abnormal surveillance data (e.g., in a de-identified form) to be forwarded to a clinical data aggregation and predictive analytics service (e.g., a clinical data warehouse and/or data store.) In certain examples, the consumer can authorize data sharing on an individual reading basis. In certain examples, the consumer can opt in for an entire electronic record with an option to opt out on an individual basis. Additionally, the consumer can share the results with his or her primary care doctor.

At block 7, the population-based predictive analytics service performs advanced analytics against the consumer's medical history, genetic profile, etc., and compares with patterns across an entire patient population, for example. The sensor and biomarker data from the self-monitoring phase are evaluated to determine if the consumer's health status has progressed compared to previous readings, for example. This analysis can be run by high-performance computers in a cloud computing environment, for example.

At block 8, a result from the computer analysis can be packaged with additional recommendations and/or guidelines and provided to the consumer. The analysis and/or additional information can be formulated in a consumer-friendly presentation rather than using professional clinician language, for example.

At block 9, the consumer is notified of discoveries and/or learning and is able to review the new information against the abnormal readings.

At block 10, the consumer can again decide whether to share the findings with a healthcare practitioner (e.g., his or her primary care doctor) and whether to seek professional counseling and/or intervention. Alternatively, the consumer's primary care doctor can be automatically notified depending upon the consumer's preference.

At block 11, if professional intervention is sought and after the doctor's assessment, the clinical data warehouse is again updated with latest findings. The doctor can review the findings from the computer-based analysis in a professional language format, for example.

At block 12, if an intervention is taken, progress throughout the treatment is updated in the clinical data warehouse.

FIG. 2 illustrates an example patient-enabled early health and preventative system 200. In certain examples, the system 200 is a cloud-delivered Mobile Computing Integration Platform as a Service (PaaS). For example, third-party sensor manufacturers and mobile computing application developers can develop applications on top of the platform or integrate with a monitoring application interface. The third party applications are then advertised in an application store and are associated with predisposed disease(s), for example. The third-party applications become available to the consumers via a portal, for example. A set of consumer-facing (e.g., patient-facing) web-based and/or mobile apps enable consumers can be provided to interact with a clearinghouse.

Example applications include entering and updating predisposal information (e.g., genetic test results, family history information, etc.); reviewing abnormal results from the self-monitoring (e.g., a results review application); consenting to share sensor data and results with clinicians and analytics data warehouse (e.g., a patient consent application); sharing information with primary care providers and other medical experts (e.g., a messaging center); reviewing and researching prevention plans and genetic predisposal research. (e.g., an information center application); etc.

In certain examples, a platform available to developers includes one or more service modules. For example, the development platform includes a sensor data processor with adapters for specific monitoring applications and sensors and programmable filters to detect abnormal sensor readings. The platform can include a terminology sub-system to normalize the sensor readings into standardized formats that are understood by the downstream analytics service, for example. The platform can include an event analyzer to analyze time series data to determine whether to alert the consumer, for example. A “patient notifier” service can be provided to alert the consumer in case of abnormal readings and/or if new information becomes available from the clinical data warehouse and/or other data source, for example. A “biomarker transmitter” can be provided that forwards standardized sensor data in an anonymous form to a clinical research cloud and predictive analytics service, for example.

In certain examples, an application store can be provided for third party application developers to advertise their applications and/or sensors. A “consumer language” translation service can be provided that can interpret clinical terminology into a consumer friendly language, for example. A “social experience” module can be included in the platform that enables consumers to rate sensors and devices in the market exchange as well as form social network groups around specific diseases and prevention plans, for example. A “professional intervention” integration module can be provided to enable consumers to link their primary care provider and other medical specialists to the sensor readings, findings, and/or recommendations from the clinical analytics service (e.g., via a clinical research cloud). In certain examples, service endpoints are provided to exchange bio-markers (e.g., sensor data), predisposal information, genetic profile, family history, etc., with the clinical research cloud. In certain examples, a knowledge base repository is provided and updated with discoveries and learning from the clinical research cloud, for example.

Using the development platform, sensors, and analytics system in conjunction with a variety of data sources can provide more advanced analytics and discovery, which in turn increases the value of those products and services. In certain examples, clinical decision support and clinical data services can interact with additional data sources by integrating with health information exchanges. In certain examples, mobile sensors (and/or home health devices) can feed data into the clinical data warehouse through smart filtering and data normalization. This, in turn, can help advance clinical research and provide new insight into effectiveness of preventive care methods and identification of unmet needs for the pharmaceutical industry, for example.

As illustrated in the example early health monitoring and analysis system 200 of FIG. 2, one or more sensors 205 are attached and/or otherwise positioned with respect to a patient 201. The sensor(s) 205 communicate with an external receiver 207, such as a smart phone and/or other electronic data receiving and transmitting device. Data collected from the sensor(s) 205 and/or additional detail input by the patient 201 is transmitted from the receiver 207 to a mobile cloud computing platform-as-a-service (PaaS) 210.

The early health PaaS 210 includes a monitoring application interface 211, which receives data from the receiver 207. The monitoring interface 211 provides the received data to a sensor data processor 214 via one or more adapters 213 designed to process and/or format the received data for the sensor data processor 214. The received data is also stored in a sensor readings data store 212.

The sensor data processor 214 processes the data according to one or more terminology mappings 216. The sensor data processor 214 identifies one or more events and/or readings in the received sensor data. The data processed by the sensor data processor 214 is filtered using one or more filters 215 to identify, for example, abnormal reading(s) 217 based on one or more criterion(-ia), parameter(s), threshold(s), etc.

Abnormal reading(s) 217 are provided to an event analyzer 218. The event analyzer 218 processes the abnormal reading(s) data 217 according to one or more criterion(-a), parameter(s), threshold(s), preference(s), etc., and provides an output to a patient notifier 219 base on the data. If the abnormal reading(s) warrant notification of the patient, the patient notifier 219 facilitates alerting and/or other notification via textual, audio, and/or video/animation notification (e.g., via the receiver 207 and/or a patient's computer, personal health record, electronic medical record, etc.).

Abnormal reading(s) 217 can also be provided to a biomarker transmitter 220, which identifies one or more biomarkers from the abnormal reading(s) data 217. The biomarker transmitter 220 can interact with an anonymizer 221 to help ensure that biomarker data is transmitted anonymously (e.g., de-identified). Anonymous biomarker information can be transmitted via one or more endpoints 222 to a clinical research cloud 230, for example.

As shown in the example of FIG. 2, a consumer language interpreter 223 interprets clinical terminology into a consumer friendly language, for example. A social experience module 224 helps enable consumers to rate sensors and devices (e.g., in a market exchange) and/or to form social network group(s) around specific diseases and/or prevention plans, for example. A professional intervention module 225 helps to enable consumers to link their primary care provider and other medical specialists to the sensor readings, findings, and/or recommendations from the clinical analytics service (e.g., via \the clinical research cloud 230).

The clinical research cloud 230 includes a plurality of analytics and repositories to store, process, and dispense clinical data and associated analysis, for example. As illustrated in the example of FIG. 2, biomarker data can be provided to predictive analytics 231, a bioinformatics repository 232, a deidentified clinical data repository 233, etc. For example, the predictive analytics 231 analyze received biomarker and/or other data to predict disease(s) and/or other condition(s) to which the patient 201 and/or a similar person may be predisposed. Data in one or more of the repositories 232, 233 can be mined, shared, and/or otherwise used by the analytics 231 and/or by an external user (e.g., an authorized user for identified data and/or a broader group of users for anonymous or de-identified data). As shown in the example of FIG. 2, data from the clinical research cloud 230 can be shared with the cloud PaaS 210 via storage in a knowledge base 226.

As illustrated in the example system 200 of FIG. 2, the PaaS 210 can include a monitoring application store 227. Via the monitoring application store 227, a patient and/or other user can identify and download (e.g., by purchasing and/or installing an application for free) one or more monitoring applications and/or sensors to facilitate patient data monitoring and/or analysis, for example. One or more third party monitoring applications 240 can be made available via the application store 227, for example.

Additionally, one or more patient-facing software as a service (SaaS) applications 250 can be provided via the application store 227, for example. Patient-facing SaaS applications 250 can include a predisposal entry application 251, a patient consent application 252, an information center 253, a messaging center 254, a results reviewer 255, a marketplace portal 256, etc. The predisposal entry application 251 allows the patient and/or another user to enter and update disease and/or other condition predisposal information (e.g., genetic test results, family history information, etc.). The patient consent application 252 facilitates obtaining patient consent to share sensor data and results with clinicians, an analytics data warehouse, and/or the clinical research cloud 230, etc. The information center 253 provides information such as prevention plans, genetic predisposal research, etc., for review and research. The messaging center 254 facilitates sharing of information with primary care providers and other medical experts, for example. The results review 255 allows a user to review abnormal results from the self-monitoring data, etc.

Based on information (e.g., sensor data, biomarkers, analysis, etc.) from the early health PaaS 210, an intervention can be scheduled with a clinician via a scheduler 260. Based on information from analyzed sensor data, patient genetics/history, data and analytics from the clinical research cloud 230, etc., the PaaS 210 can determine that an intervention for predictive planning, patient treatment, and/or other consultation is advisable or beneficial, for example. The scheduler 260 can work with the PaaS 210 and/or other clinical and/or scheduling resources to schedule one or more appointments for the patient 201 with one or more clinicians and associated equipment, for example.

Thus, certain examples provide and/or help facilitate a strong ecosystem of partners and key alliances, knowledge exchange clearinghouse services, etc., for early health and prevention. Certain examples enable a consumer to be involved and help initiate health prediction, planning, and management. Certain examples provide methods, apparatus, and systems for mobile sensor data integration, enabling a marketplace and a knowledge exchange clearinghouse for early health. Certain examples provide both a focus on individual health challenges, as well as a comprehensive and integrated ecosystem.

In certain examples, the system 200 can include and/or be in communication with one or more of a plurality of information systems. Information systems may include a radiology information system (RIS), a picture archiving and communication system (PACS), Computer Physician Order Entry (CPOE), an electronic medical record (EMR), Clinical Information System (CIS), Cardiovascular Information System (CVIS), Library Information System (LIS), and/or other healthcare information system (HIS), for example. An integrated user interface facilitating access to a patient record can include a context manager, such as a clinical context object workgroup (CCOW) context manager and/or other rules-based context manager. Components can communicate via wired and/or wireless connections on one or more processing units, such as computers, medical systems, smart phones, storage devices, custom processors, and/or other processing units. Components can be implemented separately and/or integrated in various forms in hardware, software and/or firmware, for example.

In certain examples, a patient record provides identification information, allergy and/or ailment information, history information, orders, medications, progress notes, flowsheets, labs, images, monitors, summary, administrative information, and/or other information, for example. The patient record can include a list of tasks for a healthcare practitioner and/or the patient, for example. The patient record can also identify a care provider and/or a location of the patient, for example.

In certain examples, an indication can be given of, for example, normal results, abnormal results, and/or critical results. For example, the indication can be graphical, such as an icon. The user can select the indicator to obtain more information. For example, the user can click on an icon to see details as to why a result was abnormal. In certain examples, the user may be able to view only certain types of results. For example, the user may only be eligible to and/or may only select to view critical results.

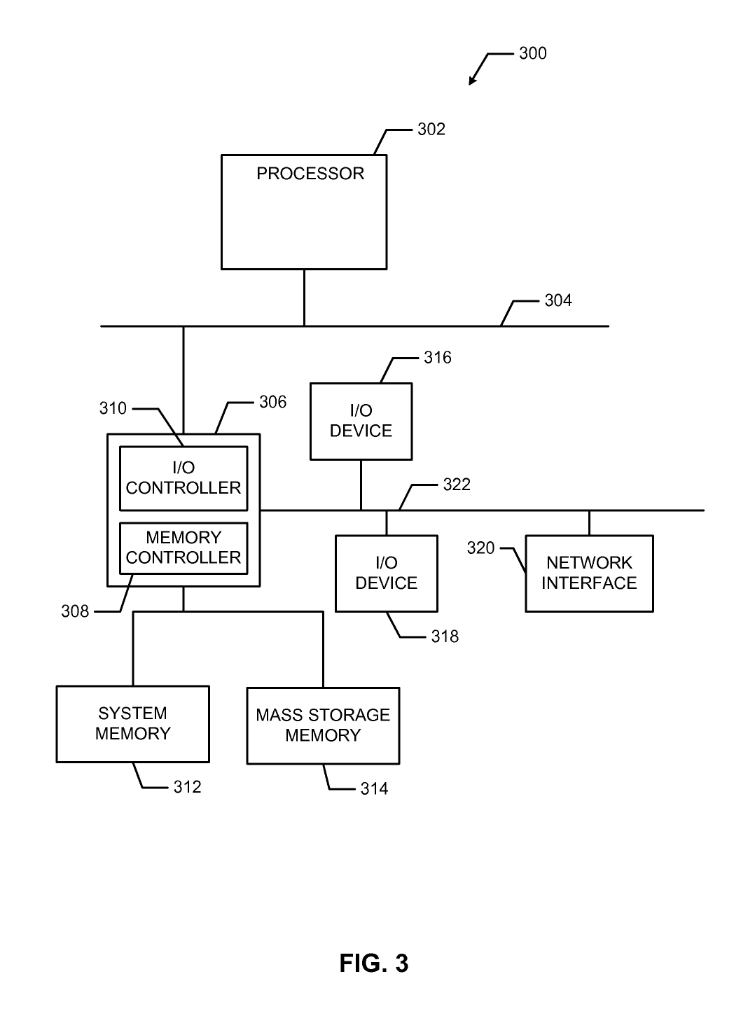

FIG. 3 is a block diagram of an example processor system 310 that can be used to implement systems, apparatus, and methods described herein. As shown in FIG. 3, the processor system 310 includes a processor 312 that is coupled to an interconnection bus 314. The processor 312 can be any suitable processor, processing unit, or microprocessor, for example. Although not shown in FIG. 3, the system 310 can be a multi-processor system and, thus, can include one or more additional processors that are identical or similar to the processor 312 and that are communicatively coupled to the interconnection bus 314. For example, “cloud” and/or “grid” based computing can be employed for three dimensional processing using Euclidian vectors and linear algebra, as described above. In certain examples, a Bayesian algorithm can be used in an evolving model combining multiple executions of multiple algorithms. As certain mappings are resolved, a probability associated with other remaining mappings changes.

The processor 312 of FIG. 3 is coupled to a chipset 318, which includes a memory controller 320 and an input/output (“I/O”) controller 322. As is well known, a chipset typically provides I/O and memory management functions as well as a plurality of general purpose and/or special purpose registers, timers, etc. that are accessible or used by one or more processors coupled to the chipset 318. The memory controller 320 performs functions that enable the processor 312 (or processors if there are multiple processors) to access a system memory 324 and a mass storage memory 325.

The system memory 324 can include any desired type of volatile and/or non-volatile memory such as, for example, static random access memory (SRAM), dynamic random access memory (DRAM), flash memory, read-only memory (ROM), etc. The mass storage memory 325 can include any desired type of mass storage device including hard disk drives, optical drives, tape storage devices, etc.

The I/O controller 322 performs functions that enable the processor 312 to communicate with peripheral input/output (“I/O”) devices 326 and 328 and a network interface 330 via an I/O bus 332. The I/O devices 326 and 328 can be any desired type of I/O device such as, for example, a keyboard, a video display or monitor, a mouse, etc. The network interface 330 can be, for example, an Ethernet device, an asynchronous transfer mode (“ATM”) device, an 802.11 device, a DSL modem, a cable modem, a cellular modem, etc., that enables the processor system 310 to communicate with another processor system.

While the memory controller 320 and the I/O controller 322 are depicted in FIG. 3 as separate blocks within the chipset 318, the functions performed by these blocks can be integrated within a single semiconductor circuit or can be implemented using two or more separate integrated circuits.

Certain embodiments contemplate methods, systems and computer program products on any machine-readable media to implement functionality described above. Certain embodiments can be implemented using an existing computer processor, or by a special purpose computer processor incorporated for this or another purpose or by a hardwired and/or firmware system, for example.

Some or all of the system, apparatus, and/or article of manufacture components described above, or parts thereof, can be implemented using instructions, code, and/or other software and/or firmware, etc. stored on a machine accessible or readable medium and executable by, for example, a processor system (e.g., the example processor system 310 of FIG. 3). When any of the appended claims are read to cover a purely software and/or firmware implementation, at least one of the components is hereby expressly defined to include a tangible medium such as a memory, DVD, CD, Blu-ray disc, etc. storing the software and/or firmware.

Certain embodiments contemplate methods, systems and computer program products on any machine-readable media to implement functionality described above. Certain embodiments can be implemented using an existing computer processor, or by a special purpose computer processor incorporated for this or another purpose or by a hardwired and/or firmware system, for example.

Certain embodiments include computer-readable media for carrying or having computer-executable instructions or data structures stored thereon. Such computer-readable media can be any available media that can be accessed by a general purpose or special purpose computer or other machine with a processor. By way of example, such computer-readable media can include RAM, ROM, PROM, EPROM, EEPROM, Flash, CD-ROM, DVD, Blu-ray or other optical disk storage, magnetic disk storage or other magnetic storage devices, or any other medium which can be used to carry or store desired program code in the form of computer-executable instructions or data structures and which can be accessed by a general purpose or special purpose computer or other machine with a processor. Combinations of the above are also included within the scope of computer-readable media. Computer-executable instructions include, for example, instructions and data which cause a general purpose computer, special purpose computer, or special purpose processing machines to perform a certain function or group of functions.

Generally, computer-executable instructions include routines, programs, objects, components, data structures, etc., that perform particular tasks or implement particular abstract data types. Computer-executable instructions, associated data structures, and program modules represent examples of program code for executing steps of certain methods and systems disclosed herein. The particular sequence of such executable instructions or associated data structures represent examples of corresponding acts for implementing the functions described in such steps.

Embodiments of the present invention can be practiced in a networked environment using logical connections to one or more remote computers having processors. Logical connections can include a local area network (LAN) and a wide area network (WAN) that are presented here by way of example and not limitation. Such networking environments are commonplace in office-wide or enterprise-wide computer networks, intranets and the Internet and can use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention can also be practiced in distributed computing environments where tasks are performed by local and remote processing devices that are linked (either by hardwired links, wireless links, or by a combination of hardwired or wireless links) through a communications network. In a distributed computing environment, program modules can be located in both local and remote memory storage devices.

While the invention has been described with reference to certain embodiments, it will be understood by those skilled in the art that various changes can be made and equivalents can be substituted without departing from the scope of the invention. In addition, many modifications can be made to adapt a particular situation or material to the teachings of the invention without departing from its scope. Therefore, it is intended that the invention not be limited to the particular embodiment disclosed, but that the invention will include all embodiments falling within the scope of the appended claims.

1. A patient preventive health system comprising:
a monitoring application interface to receive data from one or more sensors positioned with respect to a patient;
a sensor data processor to process the received data from the one or more sensors to identify one or more readings based on the received data;
an event analyzer to process the one or more readings to generate an event output;
a patient notifier to notify the patient based on the event output; and
a biomarker transmitter to identify and transmit a biomarker to a clinical research cloud based on the one or more readings.

2. The system of claim 1, further comprising one or more sensors to be positioned with respect to the patient to gather data from the patient and relay the data to a mobile receiver to be provided to the monitoring application interface.

3. The system of claim 1, wherein the sensor data processor is to process the received data according to one or more terminology mappings.

4. The system of claim 1, further comprising one or more adapters to format the received data to be processed by the sensor data processor.

5. The system of claim 1, further comprising one or more filters to identify one or more abnormal readings in the one or more readings processed by the sensor data processor.

6. The system of claim 1, further comprising an anonymizer to anonymize the biomarker for transmission to the clinical research cloud.

7. The system of claim 1, further comprising preventive analytics to analyze the biomarker to predict at least one of a disease and a condition to which the patient may be predisposed.

8. The system of claim 1, further comprising a knowledge base to receive and store information from the clinical research cloud.

9. The system of claim 1, wherein one or more of the monitoring application interface, the sensor data processor, the event analyzer, the patient notifier, and the biomarker transmitter is to be implemented as a platform as a service.

10. The system of claim 1, further comprising one or more patient-facing software as a service applications to be provided via a monitoring application store.

11. The system of claim 1, further comprising a scheduler to schedule an intervention with a clinician based on at least one of the event output and the biomarker.

12. A tangible computer readable storage medium including executable program instructions which, when executed by a computer processor, cause the computer to implement patient preventive health system comprising:
a monitoring application interface to receive data from one or more sensors positioned with respect to a patient;
a sensor data processor to process the received data from the one or more sensors to identify one or more readings based on the received data;
an event analyzer to process the one or more readings to generate an event output;
a patient notifier to notify the patient based on the event output; and
a biomarker transmitter to identify and transmit a biomarker to a clinical research cloud based on the one or more readings.

13. The computer readable storage medium of claim 12, wherein the sensor data processor is to process the received data according to one or more terminology mappings.

14. The computer readable storage medium of claim 12, further comprising one or more filters to identify one or more abnormal readings in the one or more readings processed by the sensor data processor.

15. The computer readable medium of claim 12, further comprising preventive analytics to analyze the biomarker to predict at least one of a disease and a condition to which the patient may be predisposed.

16. The computer readable medium of claim 12, further comprising a knowledge base to receive and store information from the clinical research cloud.

17. The computer readable medium of claim 12, wherein one or more of the monitoring application interface, the sensor data processor, the event analyzer, the patient notifier, and the biomarker transmitter is to be implemented as a platform as a service.

18. The computer readable medium of claim 12, further comprising one or more patient-facing software as a service applications to be provided via a monitoring application store.

19. The computer readable medium of claim 12, further comprising a scheduler to schedule an intervention with a clinician based on at least one of the event output and the biomarker.

20. A computer-implemented method for providing patient-enabled early health and preventive care, the method comprising:
suggesting, based on patient predisposal information related to at least one of a disease and a medical condition, a prevention plan for the patient;
receiving, from one or more sensors positioned with respect to the patient, data related to patient health status;
adjusting the prevention plan for the patient based on received data from the one or more sensors;
applying one or more filters to the received data to identify one or more abnormal sensor readings; and
providing, based on review and consent by the patient, data regarding the one or more abnormal sensor readings to a clinical data aggregation and predictive analytics for further processing.

21. The method of claim 20, further comprising receiving information from the patient regarding patient predisposition to the at least one of a disease and a medical condition.

22. The method of claim 20, further comprising notifying the patient of discoveries relevant to the patient's abnormal sensor readings obtained from the clinical data aggregation and predictive analytics.

23. The method of claim 20, further comprising updating information sent to the clinical data aggregation and predictive analytics based on an invention taken by a healthcare practitioner.
